(19) United States
(12) Patent Application Publication
(10) Pub. No.: US 2012/0016451 A1
(43) Pub. Date: Jan. 19, 2012

(54) TORQUE ENHANCEMENT FOR MRI-CONDITIONALLY SAFE MEDICAL DEVICE LEAD

(76) Inventors: Roger Struve, St. Paul, MN (US); Arthur J. Foster, Centerville, MN (US); James G. Bentsen, North St. Paul, MN (US)

(21) Appl. No.: 13/156,099

(22) Filed: Jun. 8, 2011

Related U.S. Application Data
(60) Provisional application No. 61/363,857, filed on Jul. 13, 2010.

(51) Int. Cl. A61N 1/05

(52) U.S. Cl. 607/116

(57) ABSTRACT

An implantable medical device lead includes an inner conductor assembly coupled to a first electrode at a distal end of the inner conductor assembly and an outer conductive coil extending coaxially with the inner conductor assembly and coupled to a second electrode. The inner conductor assembly includes one or more filars arranged in a plurality of serially connected current suppression modules. The inner conductor assembly is configured to improve torque transmission.

### CROSS-REFERENCE TO RELATED APPLICATION

This application claims the benefit under 35 U.S.C. §119(e) to U.S. Provisional Patent Application No. 61/363,857, filed on Jul. 13, 2010, entitled “TORQUE ENHANCEMENT FOR MRI-CONDITIONALLY SAFE MEDICAL DEVICE LEAD,” which is incorporated herein by reference it its entirety

### TECHNICAL FIELD

The present invention relates to implantable medical devices. More particularly, the present invention relates to an implantable medical device lead including an inner conductor having a layered coil arrangement and an outer coil.

### BACKGROUND

Magnetic resonance imaging (MRI) is a non-invasive imaging procedure that utilizes nuclear magnetic resonance techniques to render images within a patient's body. Typically, MRI systems employ the use of a magnetic coil having a magnetic field strength of between about 0.2 to 3 Teslas. During the procedure, the body tissue is briefly exposed to RF pulses of electromagnetic energy in a plane perpendicular to the magnetic field. The resultant electromagnetic energy from these pulses can be used to image the body tissue by measuring the relaxation properties of the excited atomic nuclei in the tissue. During imaging, the electromagnetic radiation produced by the MRI system may be picked up by implantable device leads used in implantable medical devices such as pacemakers or cardiac defibrillators.

### SUMMARY

Discussed herein are various conductor configurations for implantable medical electrical leads including an outer conductive coil extending coaxially with an inner multi-layer conductor assembly, as well as medical electrical leads including such conductor configurations.

Example 1 is an implantable lead that includes an inner conductor assembly extending between a distal region and a proximal region, the inner conductor assembly including one or more filars arranged in a plurality of serially connected current suppression modules, each current suppression module including a first coil of the one or more filars wound in a first winding direction, a second coil of the one or more filars coaxial with the first winding and wound in a second winding direction opposite the first winding direction, and a third coil of the one or more filars coaxial with the first and second windings and wound in the first winding direction. A stiffening agent is disposed over at least a portion of the inner conductor assembly. An outer conductive coil is disposed coaxial with the inner conductor assembly and includes one or more filars wound in the first winding direction. A flexible body extends coaxially with the outer conductive coil with an electrode disposed about the flexible body, the electrode electrically connected to the outer conductive coil. A connector assembly is secured to the proximal end of the body for coupling the lead to an implantable medical device and includes a terminal pin rotatably secured to the inner conductor assembly. A distal assembly is coupled to the distal end of the body and includes a helical electrode rotatably secured to the inner conductor assembly such that rotation of the terminal pin causes the helical electrode to rotate.

In Example 2, the implantable lead of Example 1 in which the stiffening agent includes a sheath that extends coaxially with the inner conductive assembly.

In Example 3, the implantable lead of Example 1 in which the stiffening agent includes a plurality of distinct sheath segments, at least some of the plurality of distinct sheath segments aligned with transitions between adjacent current suppression modules.

In Example 4, the implantable lead of any of Examples 1-3, further including an insulation layer disposed between the stiffening agent and the outer conductive coil.

In Example 5, the implantable lead of any of Examples 1-4, further including a low friction sheath disposed between the stiffening agent and the outer conductive coil.

In Example 6, the implantable lead of any of Examples 1-5 in which the outer conductive coil includes two or fewer filars each having a filar diameter, and wherein a pitch of the outer conductive coil is less than three times the filar diameter.

In Example 7, the implantable lead of Example 6 in which the outer conductive coil includes a single filar having a filar diameter, and wherein the pitch of the outer conductive coil is less than two times the filar diameter.

In Example 8, the implantable lead of any of Examples 1-7 in which each of the plurality of current suppression modules has a length between about 1.5 cm and about 10 cm.

In Example 9, the implantable lead of any of Examples 1-8 in which the inner conductive assembly includes four filars.

In Example 10, the implantable lead of any of Examples 1-9 in which the one or more filars of the first coil extend in a forward lengthwise direction, the one or more filars of the second coil extend in a substantially opposing reverse lengthwise direction and the one or more filars of the third coil extend in the forward lengthwise direction.

Example 11 is an implantable medical device lead that includes an inner conductor assembly coupled to a first electrode at a distal end of the inner conductor assembly, the inner conductor assembly including one or more filars arranged in a plurality of serially connected current suppression modules, each current suppression module including a first coil of the one or more filars wound in a first winding direction, a second coil of the one or more filars coaxial with the first winding and wound in a second winding direction opposite the first winding direction, and a third coil of the one or more filars coaxial with the first and second windings and wound in the first winding direction. A stiffening sheath is disposed about and extending coaxially with the inner conductor assembly. An outer conductive coil extends coaxially with the stiffening sheath and the inner conductor assembly, the outer conductive coil coupled to a second electrode and including one or more filars wound in the first winding direction.

In Example 12, the implantable medical device lead of Example 11 in which the outer conductive coil includes two or fewer filars each having a filar diameter, and wherein a pitch of the outer conductive coil is less than three times the filar diameter.

In Example 13, the implantable medical device lead of Example 12, wherein the outer conductive coil includes a single filar, and wherein the pitch of the outer conductive coil is less than two times the filar diameter.

In Example 14, the implantable medical device lead of any of Examples 11-13 in which each current suppression module has a length of between about 1.5 cm and 10 cm.

In Example 15, the implantable medical device lead of any of Examples 11-14 in which the inner conductor assembly includes four filars.

Example 16 is an implantable medical device lead that includes an insulative lead body and an outer conductive coil extending through the lead body, the outer conductive coil coupled to a proximal electrode and including one or more filars wound in a first winding direction. An inner conductor assembly extends coaxially with the outer conductive coil and is coupled to a distal electrode at a distal end of the inner conductor assembly, the inner conductor assembly including one or more filars arranged in a plurality of serially connected current suppression modules, each current suppression module including a multi-layer coil configuration with the one or more filars wound in a first coiled section having a first winding direction and extending in a forward lengthwise direction for a first forward physical length, the one or more filars then turning to merge into a proximately positioned second coiled section wound in a second winding direction opposite the first winding direction that extends in a substantially opposing reverse lengthwise direction for a reverse physical length, the one or more filars then turning to merge into a proximately positioned third coiled section wound in the first winding direction that extends in the forward lengthwise direction for a second forward physical length. A polymeric sheath is disposed over the inner conductive assembly to improve torque transmission within the inner conductive assembly.

In Example 17, the implantable medical device lead of Example 16 in which the outer conductive coil includes two or fewer filars each having a filar diameter, and wherein a pitch of the outer conductive coil is less than three times the filar diameter.

In Example 18, the implantable medical device lead of Example 17 in which the outer conductive coil includes a single filar, and wherein the pitch of the outer conductive coil is less than two times the filar diameter.

In Example 19, the implantable medical device lead of any of Examples 16-18 in which each current suppression module has a length of between about 1.5 cm and 10 cm.

In Example 20, the implantable medical device lead of any of Examples 16-19 in which the inner conductor assembly includes four filars.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a cardiac rhythm management (CRM) system including a pulse generator and a lead implanted in a patient's heart according to an embodiment of the present invention.

FIG. 2 is a side view of a lead suitable for use with the CRM system shown in FIG. 1.

FIG. 3 is a perspective view of a lead portion including an embodiment of an inner conductive assembly and an outer coil extending coaxially with the inner conductive assembly.

FIG. 4 is a cross-sectional view of the lead portion shown in FIG. 2, illustrating the series connected current suppression modules of the inner conductive assembly.

FIG. 5 is a cross-sectional view of a lead portion according to an embodiment of the present invention.

FIG. 6 is a cross-sectional view of a lead portion according to an embodiment of the present invention.

FIG. 7 is a partial cross-sectional view of a distal portion of an active fixation lead according to an embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

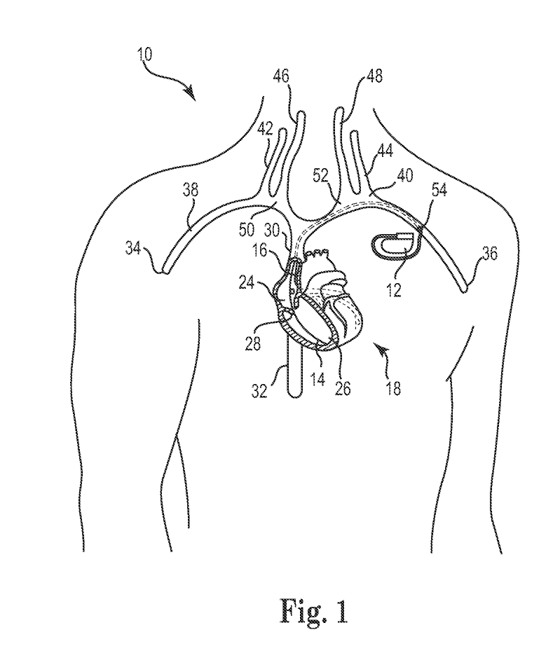

FIG. 1 is a schematic view of a cardiac rhythm management (CRM) system 10 according to an embodiment of the present invention. As shown in FIG. 1, the CRM system 10 includes a pulse generator 12 coupled to a plurality of leads 14, 16 deployed in a patient's heart 18. As further shown in FIG. 1, the heart 18 includes a right atrium 24 and a right ventricle 26 separated by a tricuspid valve 28. During normal operation of the heart 18, deoxygenated blood is fed into the right atrium 24 through the superior vena cava 30 and the inferior vena cava 32. The major veins supplying blood to the superior vena cava 30 include the right and left axillary veins 34 and 36, which flow into the right and left subclavian veins 38 and 40. The right and left external jugular 42 and 44, along with the right and left internal jugular 46 and 48, join the right and left subclavian veins 38 and 40 to form the right and left brachiocephalic veins 50 and 52, which in turn combine to flow into the superior vena cava 30.

The leads 14, 16 operate to convey electrical signals and stimuli between the heart 18 and the pulse generator 12. In the illustrated embodiment, the lead 14 is implanted in the right ventricle 26, and the lead 16 is implanted in the right atrium 24. In other embodiments, the CRM system 10 may include additional leads, e.g., a lead extending into a coronary vein for stimulating the left ventricle in a bi-ventricular pacing or cardiac resynchronization therapy system. As shown, the leads 14, 16 enter the vascular system through a vascular entry site 54 formed in the wall of the left subclavian vein 40, extend through the left brachiocephalic vein 52 and the superior vena cava 30, and are implanted in the right ventricle 26 and right atrium 24, respectively. In other embodiments of the present invention, the leads 14, 16 may enter the vascular system through the right subclavian vein 38, the left axillary vein 36, the left external jugular 44, the left internal jugular 48, or the left brachiocephalic vein 52.

The pulse generator 12 is typically implanted subcutaneously within an implantation location or pocket in the patient's chest or abdomen. The pulse generator 12 may be any implantable medical device known in the art or later developed, for delivering an electrical therapeutic stimulus to the patient. In various embodiments, the pulse generator 12 is a pacemaker, an implantable cardiac defibrillator, and/or includes both pacing and defibrillation capabilities. The portion of the leads 14, 16 extending from the pulse generator 12 to the vascular entry site 54 are also located subcutaneously or submuscularly. The leads 14, 16 are each connected to the pulse generator 12 via proximal connectors. Any excess lead length, i.e., length beyond that needed to reach from the pulse generator 12 location to the desired intracardiac implantation site, is generally coiled up in the subcutaneous pocket near the pulse generator 12.

The electrical signals and stimuli conveyed by the pulse generator 12 are carried to electrodes at the distal ends of leads 14, 16 by one or more conductors extending through the leads 14, 16. The one or more conductors are each electrically coupled to a connector suitable for interfacing with the pulse generator 12 at the proximal end of the leads 14, 16 and to one or more electrodes at the distal end. In an MRI environment, the electromagnetic radiation produced by the MRI system may be picked up by conductors of the leads 14, 16. This energy may be transferred through the leads 14, 16 to the electrode in contact with the tissue, which may lead to elevated temperatures at the point of contact. The present invention relates to a bipolar lead having an inner conductive assembly including a plurality of series connected current suppression modules that reduces heating due to MRI induced energy. The bipolar lead also includes an outer conductive coil configured to minimize the effect on the energy picked up by the inner conductive assembly.

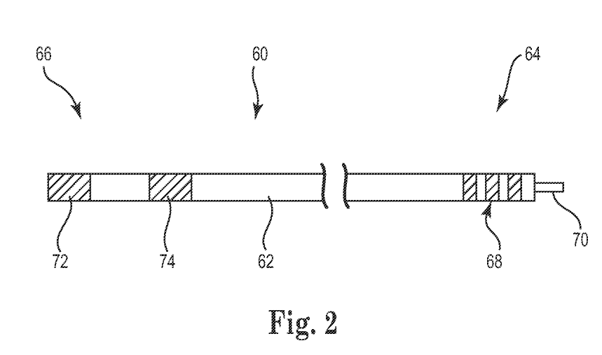

FIG. 2 is a side view of a lead 60 that may be suitable for use with the CRM system 10 shown in FIG. 1. That is, the leads 14 and/or 16 shown in FIG. 1 may be configured similarly to the lead 60. In some embodiments, the leads 14 and/or 16 may be passive fixation leads. In some embodiments, the leads 14 and/or 16 may be active fixation leads.

The lead 60 includes a flexible lead body 62 having a proximal region 64 and a distal region 66. The lead 60 includes a proximal connector 68 located within the proximal region 62. In some embodiments, as illustrated, the proximal connector 68 may include a terminal pin 70. In the illustrated embodiment, the distal region 66 of the lead 60 includes a first electrode 72 and a second electrode 74. In some embodiments, the lead 60 may include additional electrodes (not illustrated). In some embodiments, if the lead 60 is an active fixation lead, the first electrode 72 may be an electrically active fixation helix. In some embodiments, the first electrode 72 and/or the second electrode 74 may each independently be formed as coil electrodes.

In some embodiments, the lead 60 may include an inner conductive assembly that provides electrical communication between the proximal connector 68 and the first electrode 72 as well as an outer conductive coil that provides electrical communication between the proximal connector 68 and the second electrode 74. In some embodiments, if additional electrodes such as additional pace/sense electrodes or if shocking electrodes are present, the lead 60 may also include one or more high voltage conductive cables.

In some embodiments, the proximal connector 68 is configured to couple to the pulse generator 12 (FIG. 1) and electrically connects the electrodes 72, 74 to the pulse generator 12 via the inner conductor assembly 82 and outer conductive coil 78, respectively. The electrodes 72, 74 are merely illustrative, and may be configured for use in pacing, sensing, heart failure, and/or shock therapy applications. In addition, the electrode 72 may be configured for passive or active fixation of the lead 60 to tissue of the heart 18.

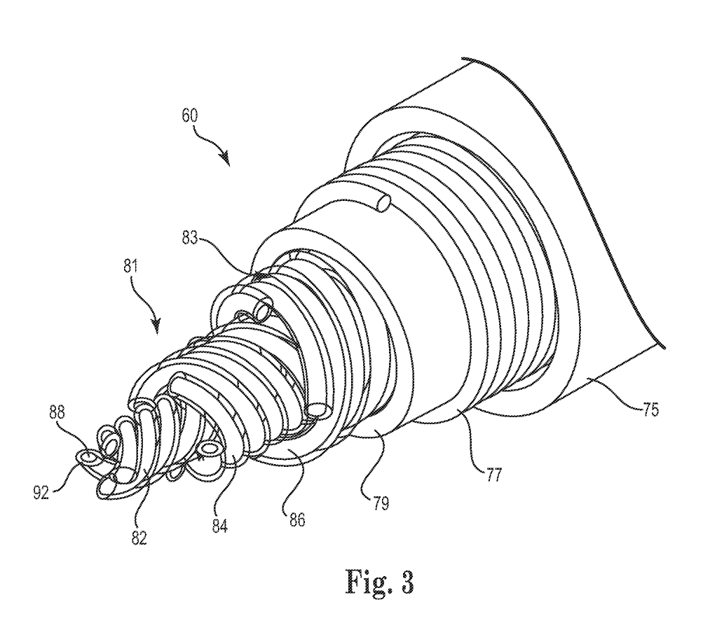

FIG. 3 is a partially cutaway perspective view of a portion of the lead 60. In this illustration, individual layers or elements are sequentially cut away to reveal the underlying structure. In FIG. 3, the lead 60 can be seen as having an outer insulative layer 75, an outer conductive coil 77, an inner insulative layer 79 and an inner conductive assembly 81. In some embodiments, the outer insulative layer 75 and/or the inner insulative layer 79 may be formed from silicon, polyurethane, or another suitable polymeric material.

As better illustrated in subsequent drawings, the lead 60 also includes a stiffening agent or layer 83 that is disposed between the inner conductive assembly 81 and the inner insulative layer 79. The lead 60 may also include one or more low friction sheaths (not illustrated) to better permit the inner conductive assembly 81 to rotate relative to the outer conductive coil 77.

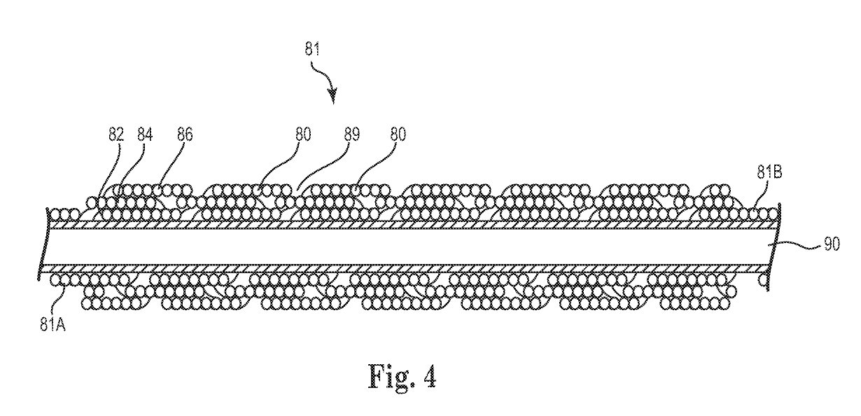

As will be described in greater detail with respect to FIG. 4, the inner conductor assembly 81 includes an inner first coil 82, an intermediate second coil 84 and an outer third coil 86. The inner first coil 82 and outer third coil 86 are wound in a first, forward direction, and the inner second coil 84 is wound in a second, reverse direction. For example, in some embodiments, the inner first coil 82 and outer third coil 86 are wound in a left hand wind (i.e., right to left relative to a proximal to distal view of the lead 60), and the intermediate second coil 84 is wound in right hand wind (i.e., left to right relative to a proximal to distal view of the lead 60). In other embodiments, the inner first coil 82 and outer third coil 86 are wound in a right hand wind, and the intermediate second coil 84 is wound in a left hand wind.

In some embodiments, the inner conductor assembly 81 includes about 2 to about 50 filars 88. In one embodiment, the inner conductor assembly 81 includes four filars 88. In some embodiments, the diameter of each filar is in the range of about 0.001 inch to 0.010 inch (0.003-0.025 cm). The filars may include biocompatible materials, including, but not limited to, Au, Ag, Nitinol, Ti, Pt, Ir, MP35N, or stainless steel. The filars may also each include an insulation layer 92 of a biocompatible and dielectric material such as, for example, Teflon, Nylon, polymers, polytetrafluoroethylene (PTFE), ethylene tetrafluoroethylene (ETFE), silicone, polyurethane, poly ether ethyl ketone (PEEK), and/or epoxy. The thickness of the insulation layer 92 may be less than about 0.005 inch (0.01 cm). In some embodiments, the outside diameter of the inner conductive assembly 70 is less than about 0.10 inch (0.25 cm).

FIG. 4 is a cross-sectional view of the inner conductor assembly 81. The inner conductor assembly 81 includes a plurality of series connected current suppression modules 80 that each include one or more filars wound into a multilayer coil assembly as will be described in more detail below. The number of current suppression modules 80 may vary depending on the desired length of the lead 60, and thus the length of the inner conductor assembly 81. In FIG. 4, there are six full current suppression modules 80 and a partial current suppression module 80. In some embodiments, there may be fewer than six current suppression modules 80. In some embodiments, there may be more than six current suppression modules. In some embodiments, for example, there may be about 10 to about 20 current suppression modules 80. The inner conductor assembly 81 may, as illustrated, include a coil winding initiation point 81A and a coil winding termination point 81B.

The segmented construction of the current suppression modules 80 prevent an MRI-induced RF standing wave from being generated on the inner conductor assembly 81. In addition, the arrangement of the filar(s) of the current suppression modules 80 cancels MRI-induced currents on the inner conductor assembly 81. Co-pending application Ser. No. 61/291,076, entitled “MRI-CONDITIONALLY SAFE MEDICAL DEVICE LEAD”, which is hereby incorporated by reference in its entirety, provides thermal data illustrating the effectiveness of the current suppression modules 80.

Each current suppression module 80 is an elongate conductor that turns back on itself at least twice in a lengthwise direction to form a conductor configuration of a reverse or backward section in one lengthwise direction and proximately located forward sections that extend in the opposing lengthwise direction. That is, the inner conductor assembly 81 is formed by winding the one or more filars into a plurality of multi-layer coiled configurations that each define a current suppression module 80. Each current suppression module 80 can be configured with a length that is a portion of the overall length of the inner conductor assembly 81. In some embodiments, the inner conductor assembly 81 is similar to the lead conductors including current suppression modules shown and described in U.S. Patent App. Pub. No. 2008/0262584, entitled “Methods and Apparatus for Fabricating Leads with Conductors and Related Flexible Lead Configurations,” and U.S. Patent App. Pub. No. 2008/0243218, entitled “MRI and RF Compatible Leads and Related Methods of Operating and Fabricating Leads,” each of which is hereby incorporated by reference in its entirety.

In some embodiments, each current suppression module 80 comprises a tri-layer configuration with three coiled segments closely stacked over each other, including the inner first coil 82, the intermediate second coil 84, and the outer third coil 86. The inner first coil 82 and outer third coil 86 are wound in a first, forward direction, and the inner second coil 84 is wound in a second, reverse direction. In some embodiments, each current suppression module 80 is wound from wire that is sufficiently plastic to enable the current suppression module 80 to at least substantially retain its shape without requiring external constraint yet is suitably hard to mechanically perform as desired. In some embodiments, the wire used to form the current suppression modules 80 may be fully annealed to about ¾ hard.

The inner conductor assembly 81 includes one or more filars 88 to form the plurality of current suppression modules 80 along the length of the inner conductor assembly 81. The filars 88 of the inner conductor assembly 81 are co-radially wound to form the inner first coil 82. The filars 88 are then wound back on themselves in the reverse direction to form the intermediate second coil 84 over the inner first coil 82. The filars 88 are then wound back on themselves again, reversing direction from the intermediate second coil 84 (i.e., in the same direction as the inner first coil 82), to form the outer third coil 86 over the intermediate second coil 84. The one or more filars 88 at the distal end of the outer third coil 86 then form a transition section 89 before forming the inner first coil 84 of the next current suppression module 80. In some embodiments, the current suppression modules 80 have a length in the longitudinal direction in the range of about 1.5 cm to about 10 cm., and the transition sections 89 have a length of between about 1.0 mm and about 3.0 mm. The length of each current suppression module 80 and transition section 89 can be controlled to optimize the current cancellation and segmentation in the inner conductor assembly 81. The current suppression modules 80 are arranged to define an inner lumen 90 and is suitable for receiving a tool to deliver the lead 60, such as a guidewire or stent.

Each of the coils 82, 84, and 86 can have a different pitch, or some or all of the coils 82, 84, and 86 can have the same pitch. In some embodiments, the inner first coil 82 can have a wider pitch and one or more of the overlying intermediate second coil 84 and outer third coil 86 can have a closer pitch.

Returning to FIG. 3, the outer conductive coil 77 is coaxially disposed about the inner conductor assembly 81 and has a helically coiled configuration that extends along all or a portion of the length of the lead 14. In some embodiments, the outer conductive coil 77 has a single-filar construction formed from a single wound wire. In other embodiments, the outer conductive coil 77 has a multifilar construction formed from multiple, co-radially wound wire filars. In one embodiment, for example, the outer conductive coil 77 has a double-filar construction formed from two co-radially wound wire filars.

The outer conductive coil 77 can be spaced radially apart from the inner conductor assembly 81, electrically isolating the outer conductive coil 77 from the inner conductor assembly 81. In some embodiments, for example, the outer conductive coil 77 is electrically isolated from the inner conductor assembly 81 so that the lead 14 can function as a multipolar lead.

In some embodiments, the outer conductive coil 77 is formed from a drawn-filled tube having an outer tubular layer of low-resistive metal or metal-alloy filled with an inner core of electrically conductive material such as silver. Once filled and drawn, the tube is then coiled into a helical shape and attached to the lead 60 using conventional techniques known in the art. In use, the relatively low resistance of the outer tubular metal or metal-alloy forming part of the outer conductive coil 77 can be used to offset the increased resistance imparted to the outer conductive coil 77 from using a smaller diameter wire. In some embodiments, the material or materials forming the outer conductive coil 77 can also be selected so as to impart greater flexibility to the outer conductive coil 77.

The outer conductive coil 77 may be formed from a material or materials different than the inner conductor assembly 81 in order to increase the resistance of the outer conductive coil 77 to aid in dissipating RF electromagnetic energy received during an MRI procedure. In one embodiment, for example, the wire filar(s) forming the outer conductive coil 77 may include a silver-filled MP35N material having a silver content (by cross-sectional area) of about 28%, whereas the wire filar(s) forming the inner conductor assembly 81 may have a silver content (by cross-sectional area) lower than 28%. In some embodiments, the filar(s) of the outer conductive coil 77 are insulated. In other embodiments, the filar(s) of the outer conductive coil 77 are not insulated.

The outer conductive coil 77 is configured to minimize the interactions and effect on energy pickup with the inner conductor assembly 81 in an MRI environment, thereby minimizing the temperature increase at the distal electrode 74. In some embodiments, the outer conductive coil 77 is wound in the same direction as the inner first coil 82 and the outer third coil 86 to minimize the interaction between the outer conductive coil 77 and inner conductor assembly 81. For example, in embodiments in which the inner first coil 82 and outer third coil 86 are wound with a left hand wind, the outer conductive coil 77 is also wound in a left hand wind. As another example, in embodiments in which the inner first coil 82 and outer third coil 86 are wound with a right hand wind, the outer conductive coil 77 is also wound with a right hand wind.

The pitch of the outer conductive coil 77 may also be minimized (i.e., closely wound) to maximize the inductance of the outer conductive coil 77, thereby making the outer conductive coil 77 more resistant to excitation in MRI fields. For example, in double filar embodiments of the outer conductive coil 77, the pitch of the outer conductive coil 77 may be about two to three times the diameter of each of the filars. In single filar embodiments of the outer conductive coil 77, the pitch of the outer conductive coil 77 may be about one to two times the filar diameter.

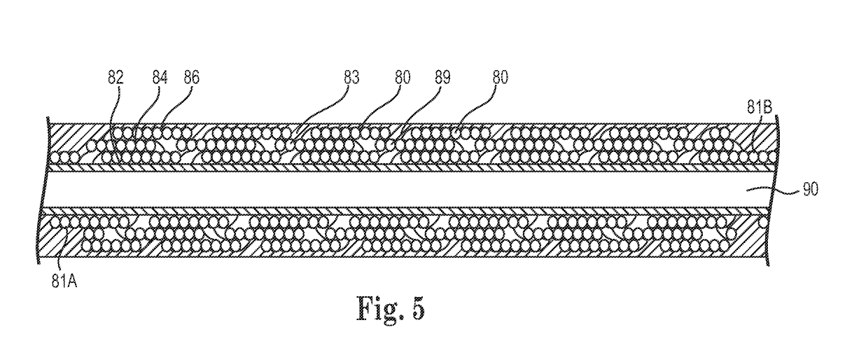

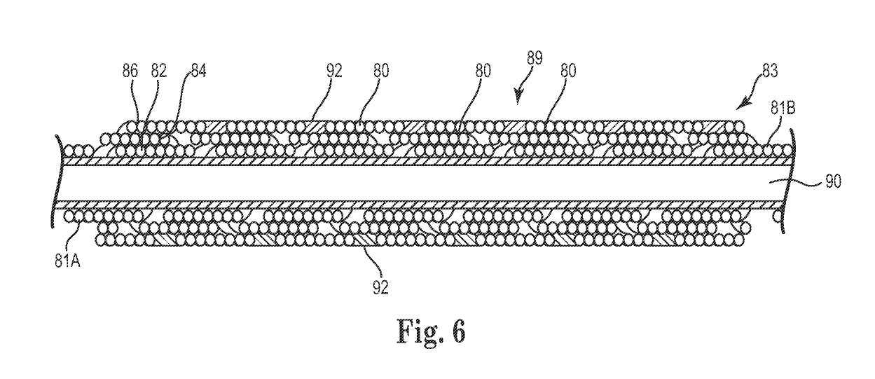

In some embodiments, as noted above, the lead 60 includes a stiffening agent or layer 83 that improves torque transmission through the inner conductor assembly 81. In some embodiments, the inner conductor assembly 81 may, without the stiffening layer 83, tend to unwind at the transitions 89 when torque is applied to the inner conductor assembly 81. The stiffening layer 83 may be formed in a variety of ways. In some embodiments, the stiffening layer 83 may extend over the length of the inner conductor assembly 81, as illustrated in FIG. 5. In some embodiments, the stiffening layer 83 may include a plurality of discrete segments that lie over the transitions 89, as illustrated in FIG. 6.

FIG. 5 is a cross-sectional view of the inner conductor assembly 81 in combination with a stiffening layer 83 that extends coaxially with the inner conductor assembly 81. In this embodiment, the stiffening layer 83 may be a polymeric layer or sheath formed of a polymer such as polytetrafluoroethylene (PTFE), polyetheretherketone (PEEK), ethylene tetrafluoroethylene (ETFE), polyurethanes, silicone rubber, polyimide, SIBBS polyurethane, and others.

In some embodiments, the stiffening layer 83 may be formed by spraying a polymer onto the inner conductor assembly 81. In some embodiments, the stiffening layer 83 may be co-extruded over the inner conductor assembly 81. In some embodiments, the stiffening layer 83 may extend over the transitions 89 without filling the transitions 89. In some embodiments, as illustrated, the stiffening layer 83 may fill in the transitions 89.

FIG. 6 is a cross-sectional view of the inner conductor assembly 81 in combination with a stiffening layer 83 that includes a plurality of distinct segments 92. In some embodiments, the distinct segments 92 may be formed from sections of shrink tubing that are heat shrunk over the transitions 89. In some embodiments, the distinct segments 92 may be formed by applying medical grade adhesive to the transitions 89. In some embodiments, the distinct segments 92 may be formed via laser or RF techniques to weld over the transitions 89.

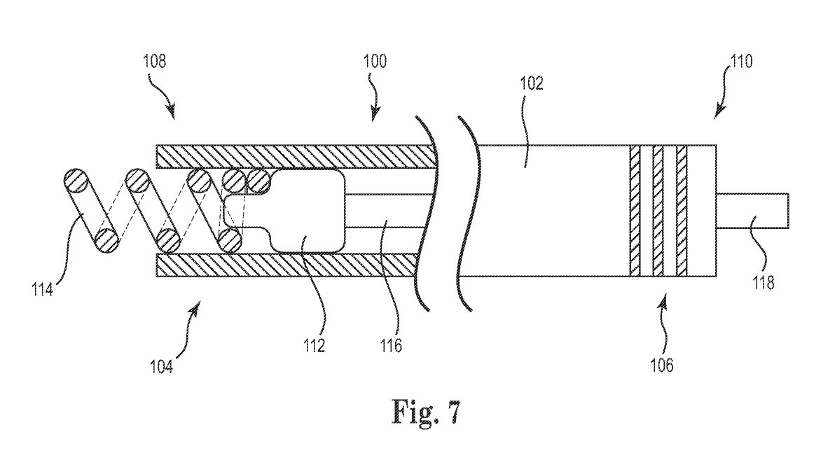

In some embodiments, as noted above, the lead 14 and/or the lead 16 may be an active fixation lead. FIG. 7 is a partial cross-sectional view of a lead 100 that may be used as one or both of the leads 14 and 16 and that may incorporate the inner conductive assembly 81 and the outer conductive coil 77 discussed above with respect to previous drawings. The lead 100 includes a lead body 102 having a distal region 104 and a proximal region 106. The distal region 104 includes a distal assembly 108 that is coupled to the lead body 102. The proximal region 106 includes a connector assembly 110 that is coupled to the lead body 12 and that is configured for connection to the pulse generator 12.

The distal assembly 108 includes an electrode base 112 to which a fixation helix 114 is attached. A conductor 116 is electrically coupled to the electrode base 112 to provide electrical connection to the electrode base 112 and thus to the fixation helix 114. The conductor 116 is also physically coupled to the electrode base 112 such that the electrode base 112 (and hence the fixation helix 114) may be rotated by rotating the conductor 116. In some embodiments, the conductor 116 is an inner conductor assembly such as the inner conductor assembly 81 described above.

The proximal assembly 110 includes a terminal pin 118. In some embodiments, the conductor 116 extends from the electrode base 112 to the terminal pin 118 and is rotatably coupled to the terminal pin 118 such that rotating the terminal pin 118 causes the conductor 116 to rotate and in turn rotate the electrode base 112 and the fixation helix 114. In some embodiments, the lead 100 may include an additional electrode (not illustrated) to which an outer conductive coil such as the outer conductive coil 77 may be electrically coupled.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable lead having a distal region and a proximal region, the implantable lead comprising:
an inner conductor assembly extending between the distal region and the proximal region, the inner conductor assembly including one or more filars arranged in a plurality of serially connected current suppression modules, each current suppression module including a first coil of the one or more filars wound in a first winding direction, a second coil of the one or more filars coaxial with the first winding and wound in a second winding direction opposite the first winding direction, and a third coil of the one or more filars coaxial with the first and second windings and wound in the first winding direction;
a stiffening agent disposed over at least a portion of the inner conductor assembly;
an outer conductive coil disposed coaxial with the inner conductor assembly, the outer conductive coil including one or more filars wound in the first winding direction;
a flexible body extending coaxially with the outer conductive coil;
an electrode disposed about the flexible body, the electrode electrically connected to the outer conductive coil;
a connector assembly secured to the proximal end of the body for coupling the lead to an implantable medical device, the connector assembly including a terminal pin rotatably secured to the inner conductor assembly; and
a distal assembly coupled to the distal end of the body, the distal assembly including a helical electrode rotatably secured to the inner conductor assembly such that rotation of the terminal pin causes the helical electrode to rotate.

2. The implantable lead of claim 1, wherein the stiffening agent comprises a sheath that extends coaxially with the inner conductive assembly.

3. The implantable lead of claim 1, wherein the stiffening agent comprises a plurality of distinct sheath segments, at least some of the plurality of distinct sheath segments aligned with transitions between adjacent current suppression modules.

4. The implantable lead of claim 1, further comprising an insulation layer disposed between the stiffening agent and the outer conductive coil.

5. The implantable lead of claim 1, further comprising a low friction sheath disposed between the stiffening agent and the outer conductive coil.

6. The implantable lead of claim 1, wherein the outer conductive coil comprises two or fewer filars each having a filar diameter, and wherein a pitch of the outer conductive coil is less than three times the filar diameter.

7. The implantable lead of claim 6, wherein the outer conductive coil comprises a single filar having a filar diameter, and wherein the pitch of the outer conductive coil is less than two times the filar diameter.

8. The implantable lead of claim 1, wherein each of the plurality of current suppression modules has a length between about 1.5 cm and about 10 cm.

9. The implantable lead of claim 1, wherein the inner conductive assembly comprises four filars.

10. The implantable lead of claim 1, wherein the one or more filars of the first coil extend in a forward lengthwise direction, the one or more filars of the second coil extend in a substantially opposing reverse lengthwise direction and the one or more filars of the third coil extend in the forward lengthwise direction.

11. An implantable medical device lead comprising:
an inner conductor assembly coupled to a first electrode at a distal end of the inner conductor assembly, the inner conductor assembly including one or more filars arranged in a plurality of serially connected current suppression modules, each current suppression module comprising a first coil of the one or more filars wound in a first winding direction, a second coil of the one or more filars coaxial with the first winding and wound in a second winding direction opposite the first winding direction, and a third coil of the one or more filars coaxial with the first and second windings and wound in the first winding direction;
a stiffening sheath disposed about and extending coaxially with the inner conductor assembly; and
an outer conductive coil extending coaxially with the stiffening sheath and the inner conductor assembly, the outer conductive coil coupled to a second electrode and including one or more filars wound in the first winding direction.

12. The implantable medical device lead of claim 11, wherein the outer conductive coil comprises two or fewer filars each having a filar diameter, and wherein a pitch of the outer conductive coil is less than three times the filar diameter.

13. The implantable medical device lead of claim 12, wherein the outer conductive coil comprises a single filar, and wherein the pitch of the outer conductive coil is less than two times the filar diameter.

14. The implantable medical device lead of claim 11, wherein each current suppression module has a length of between about 1.5 cm and 10 cm.

15. The implantable medical device lead of claim 11, wherein the inner conductor assembly comprises four filars.

16. An implantable medical device lead comprising:
an insulative lead body;
an outer conductive coil extending through the lead body, the outer conductive coil coupled to a proximal electrode and including one or more filars wound in a first winding direction;
an inner conductor assembly extending coaxially with the outer conductive coil and coupled to a distal electrode at a distal end of the inner conductor assembly, the inner conductor assembly comprising one or more filars arranged in a plurality of serially connected current suppression modules, each current suppression module including a multi-layer coil configuration with the one or more filars wound in a first coiled section having a first winding direction and extending in a forward lengthwise direction for a first forward physical length, the one or more filars then turning to merge into a proximately positioned second coiled section wound in a second winding direction opposite the first winding direction that extends in a substantially opposing reverse lengthwise direction for a reverse physical length, the one or more filars then turning to merge into a proximately positioned third coiled section wound in the first winding direction that extends in the forward lengthwise direction for a second forward physical length; and
a polymeric sheath disposed over the inner conductive assembly to improve torque transmission within the inner conductive assembly.

17. The implantable medical device lead of claim 16, wherein the outer conductive coil comprises two or fewer filars each having a filar diameter, and wherein a pitch of the outer conductive coil is less than three times the filar diameter.

18. The implantable medical device lead of claim 17, wherein the outer conductive coil comprises a single filar, and wherein the pitch of the outer conductive coil is less than two times the filar diameter.

19. The implantable medical device lead of claim 16, wherein each current suppression module has a length of between about 1.5 cm and 10 cm.

20. The implantable medical device lead of claim 16, wherein the inner conductor assembly comprises four filars.
